(19) United States
(12) Patent Application Publication
(10) Pub. No.: US 2024/0335188 A1
(43) Pub. Date: Oct. 10, 2024

(54) SYSTEM FOR AUDIO PLAYBACK CONTROL IN A SURGICAL ROBOTIC SYSTEM

(71) Applicant: Covidien LP, Mansfield, MA (US)

(72) Inventors: William J. Peine, Ashland, MA (US); Jared N. Farlow, Los Angeles, CA (US); Dustin C. Eastman, Longmont, CO (US); Barret R. Daniels, Cambridge, MA (US); Stefan Joerg, Muenchen (DE)

(21) Appl. No.: 18/293,094

(22) PCT Filed: Aug. 23, 2022
(86) PCT No.: PCT/IB2022/057901

§ 371 Date: Jan. 29, 2024

Related U.S. Application Data
(60) Provisional application No. 63/237,554, filed on Aug. 27, 2021.

(51) Int. Cl. A61B 17/00; A61B 34/00; A61B 34/37; A61B 90/00

(52) U.S. Cl. CPC A61B 17/00; A61B 34/37; A61B 34/74; A61B 90/361; A61B 2017/00119; A61B 2017/00199

(57) ABSTRACT

An audio player subsystem of a robotic surgical system is configured to play an audio file through an audio output device and control the playback of audio based on error handling and detections of the notification events. The surgical robotic system includes a robotic arm, a surgical console, a control tower, an error handler, and the audio player subsystem.

### FIELD

The present disclosure generally relates to a surgical robotic system that controls audio playback based on a surgical procedure and notification events.

### BACKGROUND

Surgical robotic systems are currently being used in minimally invasive medical procedures. Some surgical robotic systems include a surgical console controlling a surgical robotic arm and a surgical instrument having an end effector (e.g., forceps or grasping instrument) coupled to and actuated by the robotic arm.

Users in the operating room may desire to play music prior to the procedure, during the procedure, and after the procedure. The systems generate notifications to inform the users of the surgical system of the existence of any errors and other alarming or non-alarming conditions within the system or the surgical setting. In certain settings, and under certain conditions, such music playback in the operating room can be distracting, thereby diverting the attention of the users in the surgical setting away from errors and other alarming or non-alarming conditions occurring within the operating room. Therefore, it is possible for notifications to be delivered to users without the user being made aware that such notifications have been delivered.

### SUMMARY

The present disclosure provides for a surgical robotic system that controls audio playback based on a surgical procedure and notification events.

According to one aspect of the present disclosure, a surgical robotic system is disclosed and includes a robotic arm including a surgical instrument coupled thereto, a surgical console, a control tower, an error handler, and an audio player subsystem. The surgical console includes a handle communicatively coupled to the robotic arm, and a display device configured to display a user interface. The control tower is communicatively coupled to the robotic arm and the surgical console. The error handler is communicatively coupled to at least one of the robotic arm, the surgical console, or the control tower and is configured to detect a notification event. The audio player subsystem is communicatively coupled to the error handler and is configured to play an audio file through an audio output device operably coupled to the audio player subsystem, receive a detection of a notification event from the error handler, and control playback of the audio file through the audio output device based on the detection of the notification event.

In an aspect, the audio player subsystem is configured to control playback of the audio file through the audio output device based on the detection of the notification event by at least one of stopping, pausing, or muting the playback of the audio file.

In an aspect, the audio player subsystem is configured to receive a user acknowledgment of the detection of the notification event and at least one of restart, un-pause, or un-mute the playback of the audio file upon receipt of the user acknowledgment.

In an aspect, the audio player subsystem is configured to control playback of the audio file through the audio output device based on the detection of the notification event by decreasing a volume level of the playback of the audio file.

In an aspect, the audio player subsystem is configured to receive a user acknowledgment of the detection of the notification event, and increase the volume level of the playback of the audio file upon receipt of the user acknowledgment.

In an aspect, the audio output device is a directional audio output device configured to generate directional sound waves configured to be targeted in a direction of a specific user associated with the notification event.

In an aspect, the surgical robotic system includes a second audio output device operably coupled to the audio player subsystem. The audio player subsystem may be configured to control playback of the audio file to the audio output device and the second audio output device based on the detection of the notification event. Additionally, or alternatively, the audio player subsystem may be configured to control playback of the audio file through the audio output device and the second audio output device based on the detection of the notification event by adjusting the playback of the audio file through the audio output device in a first manner and adjusting the playback of the audio file through the second audio output device in a second manner different from the first manner.

In an aspect, the surgical robotic system includes an alarm and notification subsystem coupled to the error handler. The alarm and notification subsystem may be configured to display a notification on the display device based on the detection of the notification event from the error handler.

According to another aspect of the present disclosure, a surgical robotic system is disclosed and includes video analysis subsystem and an audio player subsystem. The video analysis subsystem is configured to detect a notification event within a surgical setting and detect a type of surgical procedure or a stage of the surgical procedure. The audio player subsystem is communicatively coupled to the video analysis subsystem and is configured to receive the detected type of surgical procedure or stage of the surgical procedure from the video analysis subsystem and select an audio file from a plurality of audio files to play based on at least one of the detected type of surgical procedure or stage of surgical procedure as detected by the video analysis subsystem. Additionally, the audio player subsystem is configured to play the selected audio file through an audio output device operably coupled to the audio player subsystem, receive a detection of a notification event from the video analysis subsystem, and control playback of the audio file through the audio output device based on the detection of the notification event. The audio player subsystem is configured to control playback of the audio file through the audio output device by at least one of stopping the playback of the audio file, pausing the playback of the audio file, muting a volume level of the playback of the audio file, or decreasing the volume level of the playback of the audio file.

In an aspect, the audio player subsystem is configured to receive a user acknowledgment of the detection of the notification event and control playback of the audio file through the audio output device based on the received user acknowledgment by at least one of restarting the playback of the audio file, un-pausing the playback of the audio file, un-muting the playback of the audio file, or increasing the volume level of the playback of the audio file.

In an aspect, the audio output device is a directional audio output device configured to generate directional sound waves configured to be targeted in a direction of a specific user associated with the notification event.

In an aspect, the surgical robotic system includes a second audio output device operably coupled to the audio player subsystem. The audio player subsystem may be configured to control playback of the audio file to the audio output device and the second audio output device based on the detection of the notification event. Additionally, or alternatively, the audio player subsystem may be configured to control playback of the audio file through the audio output device and the second audio output device based on the detection of the notification event by adjusting the playback of the audio file through the audio output device in a first manner and adjusting the playback of the audio file through the second audio output device in a second manner different from the first manner.

In an aspect, the surgical robotic system includes an alarm and notification subsystem configured to cause a display device to display a notification based on the detection of the notification event from the video analysis subsystem.

According to another aspect of the present disclosure, an audio player subsystem of a surgical robotic system is disclosed. The audio player subsystem is configured to play an audio file through an audio output device operably coupled to the audio player subsystem, receive priority alarm information, and control playback of the audio file through the audio output device based on the received priority alarm information. The audio player subsystem is configured to control playback of the audio file through the audio output device by at least one of stopping the playback of the audio file, pausing the playback of the audio file, muting a volume level of the playback of the audio file, or decreasing the volume level of the playback of the audio file.

In an aspect, the audio player subsystem is configured to at least one of restart, un-pause, or un-mute the playback of the audio file upon at least one of receipt of a user acknowledgment of the received priority alarm information or expiration of a preset period of time. Additionally, or alternatively the audio player subsystem may be configured to increase the volume level of the playback of the audio file upon at least one of receipt of a user acknowledgment of the received priority alarm information or expiration of a preset period of time.

In an aspect, the audio output device is a directional audio output device configured to generate directional sound waves configured to be targeted in a direction of a specific user associated with the received priority alarm information.

In an aspect, the audio player subsystem includes a second audio output device. The audio player subsystem may be configured to control playback of the audio file to the audio output device and the second audio output device based on the received priority alarm information. Additionally, or alternatively, the audio player subsystem may be configured to control playback of the audio file through the audio output device and the second audio output device based on the received priority alarm information by adjusting the playback of the audio file through the audio output device in a first manner and adjusting the playback of the audio file through the second audio output device in a second manner different from the first manner.

In an aspect, the surgical robotic system includes a video analysis subsystem configured to detect a stage of the surgical procedure. In an aspect, the audio player subsystem is configured to control playback of audio based on the detected stage of the surgical procedure.

### BRIEF DESCRIPTION OF THE DRAWINGS

Various embodiments of the present disclosure are described herein with reference to the drawings, wherein:

FIG. 1 is a schematic illustration of a surgical robotic system including a control tower, a console, and one or more surgical robotic arms according to an embodiment of the present disclosure;

FIG. 2 is a perspective view of a surgical robotic arm of the surgical robotic system of FIG. 1 according to an embodiment of the present disclosure;

FIG. 3 is a perspective view of a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a schematic diagram of an error handler subsystem according to the present disclosure;

FIG. 6 is a flow chart of a method for handling error signals according to the present disclosure; and

FIG. 7 is a flow chart of a method for controlling audio playback according to the present disclosure.

### DETAILED DESCRIPTION

The term “application” may include a computer program designed to perform functions, tasks, or activities for the benefit of a user. Application may refer to, for example, software running locally or remotely, as a standalone program or in a web browser, or other software which would be understood by one skilled in the art to be an application. An application may run on a controller, or on a user device, including, for example, a mobile device, an IOT device, or a server system.

As will be described in detail below, the present disclosure is directed to a surgical robotic system, which includes a surgical console, a control tower, and one or more movable carts having a surgical robotic arm coupled to a setup arm. The surgical console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which would, in turn, move the robotic arm in response to the movement command.

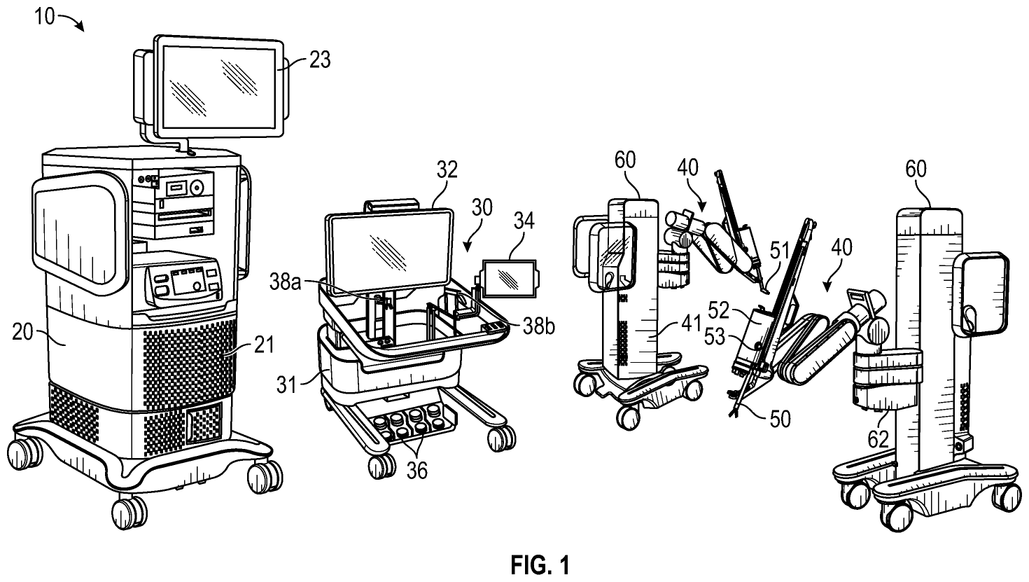

With reference to FIG. 1, a surgical robotic system 10 includes a control tower 20, which is connected to all of the components of the surgical robotic system 10 including a surgical console 30 and one or more robotic arms 40. Each of the robotic arms 40 includes a surgical instrument 50 removably coupled thereto. Each of the robotic arms 40 is also coupled to a movable cart 60.

The surgical instrument 50 is configured for use during minimally invasive surgical procedures. In embodiments, the surgical instrument 50 may be configured for open surgical procedures. In embodiments, the surgical instrument 50 may be an endoscope configured to provide a video feed for the user. In further embodiments, the surgical instrument 50 may be an electrosurgical forceps configured to seal tissue by compression tissue between jaw members and applying electrosurgical current thereto. In yet further embodiments, the surgical instrument 50 may be a surgical stapler including a pair of jaws configured to grasp and clamp tissue whilst deploying a plurality of tissue fasteners, e.g., staples, and cutting stapled tissue.

Each of the robotic arms 40 may include a camera 51 configured to capture video of the surgical site. The camera 51 may be a stereoscopic camera and may be disposed along with the surgical instrument 50 on the robotic arm 40. The surgical console 30 includes a first display 32, which displays a video feed of the surgical site provided by camera 51 of the surgical instrument 50 disposed on the robotic arms 40, and a second display device 34, which displays a user interface for controlling the surgical robotic system 10. The surgical console 30 also includes a plurality of user interface devices, such as foot pedals 36 and a pair of handle controllers 38a and 38b which are used by a user to remotely control robotic arms 40.

The control tower 20 includes a display 23, which may be a touchscreen, and outputs on the graphical user interfaces (GUIs). The control tower 20 also acts as an interface between the surgical console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgical console 30, in such a way that robotic arms 40 and the surgical instrument 50 execute a desired movement sequence in response to input from the foot pedals 36 and the handle controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area networks, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 802.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

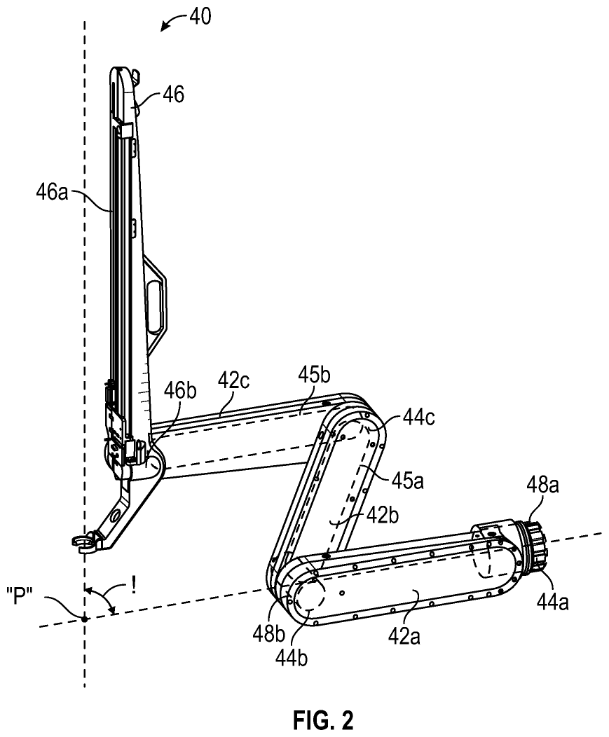

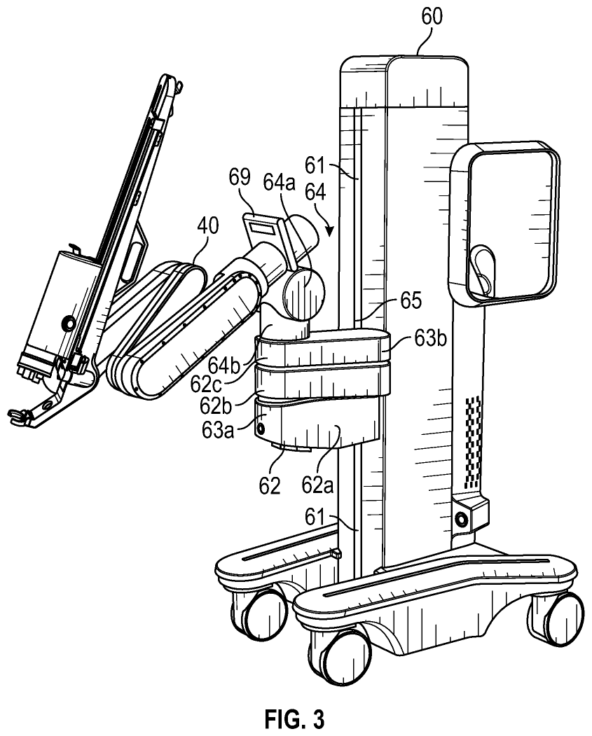

With reference to FIG. 2, each of the robotic arms 40 may include a plurality of links 42a, 42b, 42c, which are interconnected at joints 44a, 44b, 44c, respectively. The joint 44a is configured to secure the robotic arm 40 to the movable cart 60 and defines a first longitudinal axis. With reference to FIG. 3, the movable cart 60 includes a lift 61 and a setup arm 62, which provides a base for mounting of the robotic arm 40. The lift 61 allows for vertical movement of the setup arm 62. The movable cart 60 also includes a display 69 for displaying information pertaining to the robotic arm 40.

The setup arm 62 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 62 includes controls 65 for adjusting movement of the links 62a, 62b, 62c as well as the lift 61.

The third link 62c includes a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The robotic arm 40 also includes a plurality of manual override buttons 53 disposed on instrument drive unit 52 and the setup arm 62, which may be used in a manual mode. The user may press one or the buttons 53 to move the component associated with the button 53.

With reference to FIG. 2, the robotic arm 40 also includes a holder 46 defining a second longitudinal axis and configured to receive the instrument drive unit 52 (FIG. 1) of the surgical instrument 50, which is configured to couple to an actuation mechanism of the surgical instrument 50. Instrument drive unit 52 transfers actuation forces from its actuators to the surgical instrument 50 to actuate components (e.g., end effectors) of the surgical instrument 50. The holder 46 includes a sliding mechanism 46a, which is configured to move the instrument drive unit 52 along the second longitudinal axis defined by the holder 46. The holder 46 also includes a joint 46b, which rotates the holder 46 relative to the link 42c.

The robotic arm 40 also includes a plurality of manual override buttons 53 disposed on the IDU 52 and the setup arm 62, which may be used in a manual mode. The clinician may press one or the buttons 53 to move the component associated with the button 53.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

The actuator 48b of the joint 44b is coupled to the joint 44c via the belt 45a, and the joint 44c is in turn coupled to the joint 46c via the belt 45b. Joint 44c may include a transfer case coupling the belts 45a and 45b, such that the actuator 48b is configured to rotate each of the links 42b, 42c and the holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

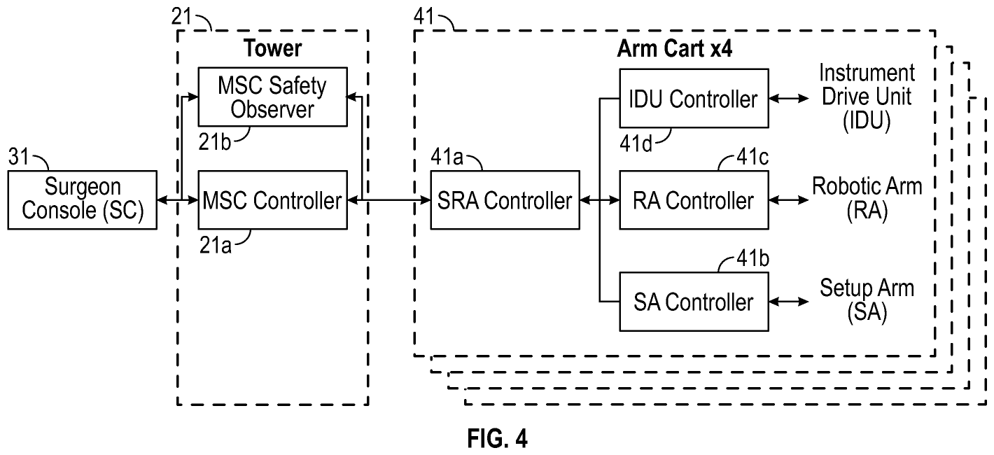

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the handle controllers 38a and 38b and the state of the foot pedals 36 and other buttons. The controller 21a processes these input positions to determine desired drive commands for each joint of the robotic arm 40 and/or the instrument drive unit 52 and communicates these to the computer 41 of the robotic arm 40. The controller 21a also receives back the actual joint angles and uses this information to determine force feedback commands that are transmitted back to the computer 31 of the surgical console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the instrument drive unit 52. The main cart controller 41a also communicates actual joint angles back to the controller 21a.

The setup arm controller 41b controls each of joints 63a and 63b, and the rotatable base 64 of the setup arm 62 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the instrument drive unit 52. The IDU controller 41d calculates actual angles based on the motor positions and transmits the actual angles back to the main cart controller 41a.

The robotic arm 40 is controlled as follows. Initially, a pose of the handle controller controlling the robotic arm 40, e.g., the handle controller 38a, is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controller 38a may be embodied as a coordinate position and role-pitch-yaw (“RPY”) orientation relative to a coordinate reference frame, which is fixed to the surgical console 30. The desired pose of the surgical instrument 50 is relative to a fixed frame on the robotic arm 40. The pose of the handle controller 38a is then scaled by a scaling function executed by the controller 21a. In embodiments, the coordinate position is scaled down and the orientation is scaled up by the scaling function. In addition, the controller 21a also executes a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

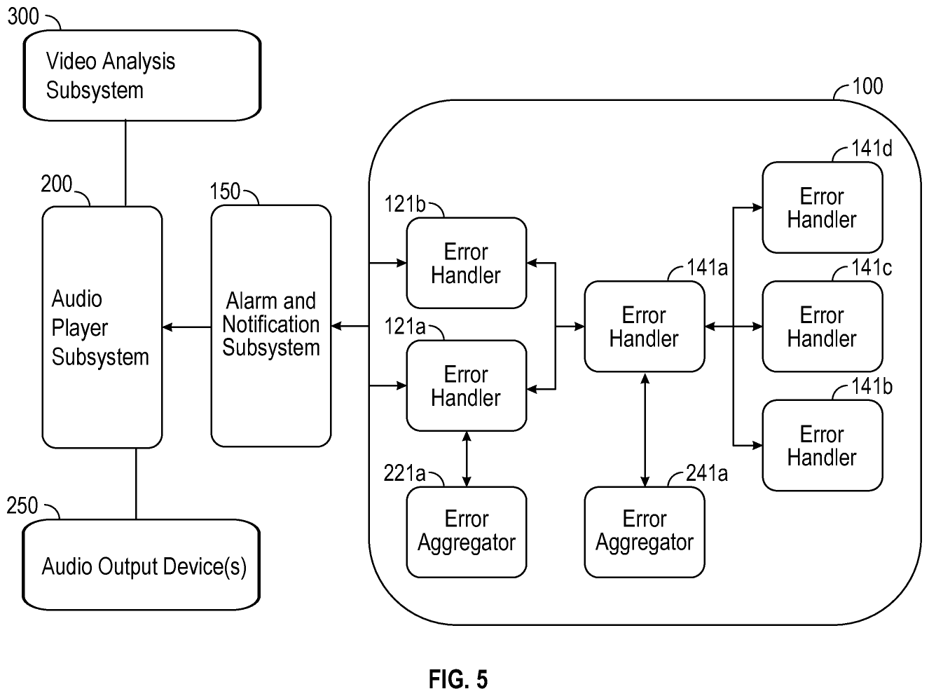

With reference to FIG. 5, the present disclosure provides an audio player subsystem 200 which operates in conjunction with an alarm and notification subsystem 150 and an error handler network 100. The audio player subsystem 200 may be part of the control tower 20, surgical console 30, and/or robotic arms 40, or may be a standalone component that is communicatively coupled to the surgical robotic system 10. The audio player subsystem 200 may be hard wired or communicably coupled to one or more audio output devices 250 (e.g., speakers) within the surgical setting to play audio (e.g., music) from any source including but not limited to a podcast, music player, show, etc. The source of the audio files/selection of audio files to play may live on a user device (e.g., smartphone or other external audio player) that is wirelessly connected to the audio player subsystem 200. The output devices may be part of the surgical robotic system 10 or standalone devices communicatively coupled to the audio player subsystem 200 of the surgical robotic system 10. The output devices 250 may also include wearable devices, such as headphones, or portable speakers. The audio output devices 250 may be a component of one or more of the control tower 20, surgical console 30, robotic arms 40, or may be a separate component such as standalone speakers, headsets, or the like, and may be configured to output directional soundwaves directed at one or more specific users in the operating room. In one aspect, one audio output device 250 may be dedicated to one user or one group of users while a different audio output device 250 may be dedicated to another user or another group of users.

The audio player subsystem 200 controls the playback of audio (e.g., music) during the surgical procedure, for example, by adjusting or modifying playback of audio (controlling the playback speed, volume, tone, pausing, or stopping, etc.) and/or by selecting which audio file to play based on the user that is operating the system, errors detected within the surgical robotic system 10 (also referred to herein as “notification events”), delivery of notifications, the type of surgical procedure, and/or progression of the surgical procedure. The audio player subsystem 200 may automatically select the audio to play based on different inputs or factors or the audio play subsystem 200 may play audio based on received user input or selection. In one embodiment, the audio play subsystem 200 is configured to select which audio file to play and may adjust the playback depending on a variety of factors selected by a user or automatically selected by the surgical robotic system 10, for example, based on the type of procedure, the current stage of the procedure, and/or other factors. For example, for procedures, or during a certain stage of a procedure that requires relatively higher concentration by a user, the audio player subsystem 200 may select to play a song, or a portion of a song, that promotes concentration and/or may adjust the playback of the audio in a manner that prevents the audio playback from distracting the user (e.g., slow the playback speed, reduce the volume, pause or stop the playback, etc.). In contrast, during another procedure, or a different stage of the procedure that requires relatively lower concentration by a user (e.g., during setup or clean-up), the audio player subsystem 200 may select to play a song or a portion of a song that promotes rapid movement by the users and/or may adjust the playback of the audio in a manner that promotes faster movement by the users (e.g., increase the playback speed, increase the volume, etc.). In an aspect, the audio player subsystem 200 monitors the progress and phase of the procedure during the course of the procedure and adjusts the tone of the audio playback (e.g., the music) based on the current stage or type of the procedure. The audio player subsystem 200 may automatically adjust or modify the playback of audio based on different inputs or factor or the audio play subsystem 200 may adjust or modify the playback of audio based on received user input or selection.

The audio player subsystem 200 may include a memory which stores a plurality of audio files for playback. In some embodiments, the audio files may be stored remotely on a server, a cloud service, or a music streaming service.

In an aspect, the audio player subsystem 200 may include input devices (e.g., microphones, image devices, laparoscopic cameras, sensors, etc.) configured to monitor stress levels of users in the operating room and upon detecting elevations or changes in stress levels within the operating room, may adjust the audio playback based on the elevated stress levels of the users or participants. In one particular example, the audio player subsystem 200 monitors the voices of participants on the operating room and detects elevated stress levels based on the voices. In further embodiments, the audio player subsystem 200 may receive surgical phases, procedures warnings, notification events, or surgical events from a surgical video analysis subsystem 300. In this aspect, the audio play subsystem 200 may adjust or select the audio playback based on the received surgical events, surgical warnings, or surgical phases. For example, the tempo or type of the music selected may vary based on if it is the beginning, middle, or ending of the procedure. The surgical video analysis subsystem 300 may be part of the control tower 20, surgical console 30, and/or robotic arms 40, or may be a standalone component.

In addition to operating based on detected stages of the surgical procedure and other signals received from the surgical video analysis subsystem 300, the audio player subsystem 200 may additionally or alternatively operate in conjunction with the alarm and notification subsystem 150 and/or the error handler network 100 to modify the audio playback based on detected notification events. A notification event, for example, may be a detected surgical event, a detected surgical phase, a detected surgical error, a detected issue that requires an alert or alarm, or any detected surgical or robotic event that may benefit from a notification to the user. For example, the notification may alert any relevant users (or all users) to the existence of an error or any alarm-type or non-alarm-type condition. The modification of the audio playback by the audio player subsystem 200, originating from detected notification events, may correspond to the type (e.g., the priority) of error or notification event detected and may be associated with (e.g., be relevant to) only select users within the surgical setting. Therefore, depending on the type of error or notification event detected, while audio player subsystem 200 may adjust the audio playback for one specific relevant user in a first manner (e.g., mute playback volume for the user) to indicate to the relevant user that the corrective action is required of the relevant user, the audio player subsystem 200 may adjust the audio playback for other non-relevant users in a second, different, manner (e.g., reduce playback volume) to make the non-relevant users aware that another individual within the surgical setting is responsible for administering a corrective action. For example, for a high priority event (e.g., one that may require the attention of all of the individuals within the surgical setting), the audio player subsystem 200 may be configured to mute, decrease the playback volume, or stop the playback of all of the audio to all of the users within the surgical setting, and in contrast, for a low priority event (e.g., one that may require the attention of only one or more users within the surgical setting), the audio player subsystem 200 may be configured to mute, decrease the playback volume, or stop the audio playback to only those relevant users.

The error handler network 100 may include any number of error handlers, which may be embodied as software executable by one or more corresponding controllers. In one example embodiment, the error handler network 100 includes error handlers 121a, 121b, 141a, 141b, 141c, 141d, which are embodied as software executable by each of the corresponding controller: the controller 21a, the safety observer 21b, the main cart controller 41a, the setup arm controller 41b, the robotic arm controller 41c, and the IDU controller 41d, respectively. The error handler network 100 is a virtual network for transmitting signals communicating between multiple subsystems (e.g., controllers) to transmit information about errors and desired system reactions. Thus, if an error occurs at one component, then a controller (e.g., the main cart controller 41a) associated with that component reacts to the error with a preprogrammed response (e.g., preprogrammed action that places the component in a safe state). The controller (e.g., main cart controller 41a) also reports the error to other subsystems such as the safety observer 21b, the main cart controller 41a, the setup arm controller 41b, and the alarm and notification subsystem 150 which operates in conjunction with the audio player subsystem 200 to alert or otherwise notify the applicable user(s) of an event, when appropriate. These subsystems then respond by initiating related safe behaviors. Safety behavior may also define an error response that includes a hard stop, e.g., complete shutdown of the surgical robotic system 10, in which all modes (including manual control mode) are disabled for the remainder of the procedure until deactivation of the surgical robotic system 10.

The error handlers 121a-b and 141a-d are networked together to coordinate a response across the surgical robotic system 10 and subsystem level in upstream/downstream and parent/child manner. Child subsystems (or child processes) refer to downstream, lower-level subsystems such as the robotic arm controller 41c, the setup arm controller 41b, and IDU controller 41d. Mid-level subsystems refer to the main cart controller 41a of each of the movable carts 60. The top-level subsystem is the controller 21a. Thus, all other error handlers 141a-141d are downstream from the error handler 121a and error handler 141a is the parent of the error handlers 141b-141d. The computer 31 of the surgical console 30 may also include a controller having an error handler, which is downstream of the error handler 121a and operates in the similar manner as the error handlers 141a-141d.

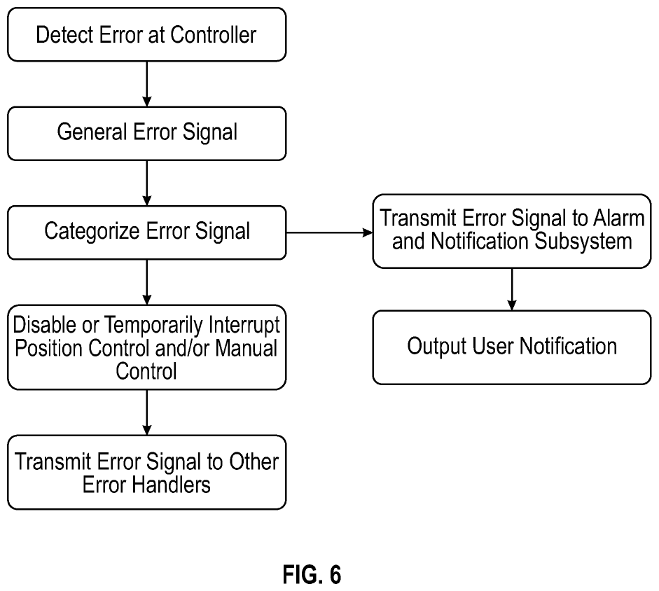

With reference to FIG. 6, initially, the error signals reported by the error handlers 121a-b and 141a-141d are categorized as an operable error or an inoperable error. As used herein, an operable error is an error during which normal function of the controllers (e.g., position control of the robotic arm controller 41c) is not affected and the user is simply notified that an error was detected. Inoperable error is an error in response to which position control of the robotic arm 40 and the surgical instrument 50 is disabled and manual control is also interrupted. Each of the controllers 21a and 41a-d reacts to the error signal based on the type of error signal. The reaction may have a predetermined duration for displaying a notification and/or a predetermined duration of how long an operational mode is disabled.

Responses to inoperable errors cause manual control of the robotic arms 40 and/or the surgical instruments 50 to be interrupted without permanently disabling the robotic arms 40 and the surgical instruments 50. Thus, if the user is actively using manual control when an inoperable error occurs then that instance of manual control is ended, and the user can let go of the button 53 and then reactivate the button 53 to re-enter manual control.

The error signals reported by the error handlers 121a-b and 141a-141d may further be classified as recoverable or nonrecoverable and as relevant to one or more specific users within the surgical setting such that actions by the audio player subsystem 200 (e.g., volume reduction, volume mute, stop or pause audio play, modify speed or tone of audio play, etc.) and actions by the alarm and notification subsystem 150 (e.g., notifications delivery) associated with the error are only imparted upon the specific relevant user or users. Recoverability for a given error is defined at both the system level and the subsystem level. In embodiments, the error is defined as affecting the entire surgical robotic system 10 and one of the components, e.g., the movable cart 60. A system nonrecoverable error denotes that the entire surgical robotic system 10 cannot be recovered back to a usable state. A subsystem nonrecoverable error denotes that only one or more of the components where the error occurred cannot be recovered back to a usable state.

When a nonrecoverable error is encountered, manual control is temporarily interrupted and position control is disabled until the surgical robotic system 10 or specific component thereof is restarted. In one example implementation, when a nonrecoverable error is encountered, the audio player subsystem 200 stops playback of the audio until the surgical robotic system 10 or specific component thereof is restarted. When a recoverable error is encountered, manual control is interrupted and position control is disabled only for a period of time, which is based on whether the error is transient or persistent, and whether the error is dismissible. In one example implementation, when a recoverable error is encountered, the audio player subsystem 200 reduces the volume of the audio playback for a period of time, which is based on whether the error is transient or persistent, and whether the error is dismissible.

Inoperable recoverable errors further include two categories: transient-type errors and persistent-type errors. Transient-type recoverable errors are further segregated into transient and dismissible errors. Persistent-type errors include nonrecoverable, persistent recoverable, and persistent-to-dismissible recoverable errors. Additionally, any of the transient-type errors or persistent-type errors, whether recoverable or non-recoverable, may be categorized as user-specific, that is, as relevant to one or more specific users within the surgical setting. Thus, the errors may be classified into the following types:
Nonrecoverable inoperable error—the affected subsystem notifies the user, adjusts the playing audio, disables position control, and interrupts manual control. Position control is disabled until the surgical robotic system 10 or specific component thereof is restarted. In one example implementation, when a nonrecoverable inoperable error is encountered, the audio player subsystem 200 stops playback of the audio until the surgical robotic system 10 is restarted.
Transient recoverable inoperable error—the affected subsystem notifies the user, adjusts the playing audio, interrupts position control, and interrupts manual control. All modes are available immediately after the error has occurred. In one example implementation, when a transient recoverable inoperable error is encountered, the audio player subsystem 200 reduces the playback volume of the audio for a period of time.
Persistent recoverable inoperable error—the affected subsystem notifies the user, adjusts the playing audio, disables position control, and interrupts manual control. Position control is disabled until the reason for the error is resolved (e.g., by user intervention). In one example implementation, when a persistent recoverable inoperable error is encountered, the audio player subsystem 200 reduces the playback volume of the audio until the reasons for the error is resolved (e.g., by user intervention).
Transient dismissible recoverable inoperable error—the affected subsystem notifies the user, adjusts the playing audio, disables position control, and interrupts manual control. Position control is disabled until the user acknowledges the notification, regardless of whether the reason for the error disappears. In one example implementation, when a transient dismissible recoverable inoperable error is encountered, the audio player subsystem 200 reduces the playback volume of the audio until the user acknowledges the notification, regardless of whether the reason for the error disappears, and then increases the playback volume once the user acknowledges the notification.
Persistent dismissible recoverable inoperable error—the affected subsystem notifies the user, adjusts the playing audio, disables position control, and interrupts manual control. Position control is disabled until the reason for the error disappears and the user acknowledges the notification. In one example implementation, when a persistent dismissible recoverable inoperable error is encountered, the audio player subsystem 200 reduces the playback volume of the audio until the user acknowledges the notification and the reason for the error disappears, and then increases the playback volume once the user acknowledges the notification and the reason for the error disappears.

With reference to FIG. 5, each controller 21a and 41a-d uses a corresponding error handler 121a-b and 141a-d to communicate with other error handlers of the other controllers as well as with the state machines in its own controller. When an error occurs in a subsystem, that subsystem reacts by disabling/interrupting control modes and then sending messages to a neighboring subsystem via the error handler. The robotic arm controller 41c, the setup arm controller 41b, and IDU controller 41d communicate error signals to the main cart controller 41a, which then replies with confirmation of receipt and control commands. The main cart controller 41a also reports error information up to the controller 21a, which then replies with confirmation of receipt and nominal control commands as well. This communication combined with the designated recovery type affects the timing of when the controllers 41b-d can resume nominal functionality.

After detecting a problem, the error handler 141b, disables position control, and sends an alarm to an alarm and notification subsystem (ANS) 150, which may be embodied as a software application and is executed by the controller 21a. The ANS 150 is coupled to various inputs and outputs of the surgical robotic system 10 and displays various audio and visual alarm and notifications on one or more of the displays 23, 32, 34, 69. The ANS 150 operates in conjunction with the audio player subsystem 200 to control the audio (e.g., music) being played by the audio player subsystem 200 based on the type (e.g., priority) of the error or notification event detected. The alarm and notification subsystem 150 also uses audio to indicate/display notifications. In one example, instead of just lowering the volume or stopping the playback, the audio player subsystem 200 plays the notification-specific audio. Additionally, or alternatively, when the playback of the user audio is not stopped, it could mix in (with increasing volume) the playback of the navigation or other audio.

The error handler 141b then sends the error signal to the error handler 141a, which then sends a confirmation of receiving the error signal and initiates a hold protocol and stops position control. Error signals are also sent to the controller 21a, which recognizes the system-wide inoperable error and interrupts any robotic arms 40 in position control or manual control. The controller 21a also sends hold commands to all robotic arms 40 and sends a confirmation of receiving an error signal to the robotic arm 40. Additionally, the ANS 150 and the audio player subsystem 200 are also independently aware of the type of the error or other notification event (e.g., that the error is a system persistent dismissible type) and deliver notifications and adjust audio playback, respectively, to the relevant user(s) based on the type of error or other notification event. Depending on the type of error or other notification event, the controller 21a can stop position control on any robotic arms 40 until the user dismisses the notification, and the user cannot dismiss the notification until the original problem is resolved.

Thus, all robotic arms 40 are locked out of position control as well as manual control. A notification appears on GUIs one or more of the displays 23, 32, 34, 69 with a graphical “dismiss” button to dismiss the error and the audio playing by the audio player subsystem 200 may be adjusted based on the type and priority of the error or other notification event, for example to bring the user's attention to the error, the notification, or the state of the error. A bedside assistant can re-activate the button 53 to get back into manual control. However, the user is not able to get back into position control because the error was a persistent dismissible type. After the problem responsible for the error independently disappears, the user can then dismiss the notification, and then the user is able to resume position control and the audio playback by the audio player subsystem 200 resumes to the preadjusted levels.

The error handler network 100 includes the following conditions, which are mapped to specific error states: manual mode for one robotic arm 40 is unavailable, position control for one robotic arm 40 is unavailable, position, manual, and hold modes for one robotic arm 40 are unavailable, and position control for all robotic arms 40 is unavailable. Conditions are mapped to the set of error states based on whether the error affects a subsystem, the surgical robotic system 10, or both. Mapping also depends on error operability and recoverability. Some error states are set while the condition is true, while other error states are set as true when the condition occurs.

As described above, inoperable recoverable errors are categorized into two categories: transient-type and persistent-type. Transient-type recoverability includes transient and dismissible errors. Persistent-type errors include nonrecoverable, persistent recoverable, and persistent to dismissible recoverable errors. In the transient-type inoperable errors, manual control and position control, as well as position control of all robotic arms 40, are interrupted but not disabled. In the persistent-type inoperable errors only manual control is interrupted and position control on all robotic arms 40 is disabled. The error state mapping for the system and subsystem persistent dismissible recoverable inoperable errors is the same as for persistent-type inoperable errors.

Regarding nonrecoverable errors, certain conditions are mapped to system and/or subsystem nonrecoverable errors. Nonrecoverable signals indicate when a nonrecoverable error has occurred on the subsystem. Each error signal generated by an error handler has its own signal pair, where the first error signal indicates subsystem (local) position control is nonrecoverable and the second error signal indicates system (global) position control is nonrecoverable. Nonrecoverable signals are set to being true during error state mapping and are transmitted upwards to parent subsystems. In the parent error handler (e.g., error handler 121a or 141a), nonrecoverable signals are analyzed by combining all of the nonrecoverable signals using an “or” operator (e.g., error handlers 141b-d as well as parent's nonrecoverable signals). If activated, these signals are latched until system deactivation.

The error handler network 100 also stores interrupt counters which when exceeded interrupt operation of the surgical robotic system 10 and/or any of the robotic arms 40. This allows the error handler network 100 to interrupt operation of the surgical robotic system 10 even when error states are dismissed. The error handlers 141a-d have a unique subsystem interrupt counter signal and a system interrupt counter. The counters represent how many errors have occurred in that subsystem. The counters are increased while the specific controller 41a-d is running continuously. The counters are reset once the specific controller 41a-d is shut down.

When an error occurs, that subsystem's error handler 141b-d increments the counter(s) and sends the signal upwards to the error handler 141a and/or the error handler 121a. The error handlers 121a and 141a detect when the counters are incremented and send interrupt signals to their state machines. The incremented counters are then sent to the child subsystems, e.g., the error handlers 141b-d. This confirmation transmission forms a cycle, which enables the child error handlers 141b-d to confirm that the messages were received by the upstream error handlers 121a and 141a.

The error confirmation cycle is used to end an active interruption of position control and/or manual control of the robotic arm 40. When any control mode is interrupted, error handlers 121a and 141a latch the error states as true until the downward counter matches the upwards counter. Error states may remain true for another reason (e.g., a persistent error), but the interrupt is ended.

The error handlers 121a may also verify the system interrupt counters. If a condition in a child error handler 141b-d maps to a system level inoperable error, the system interrupt counter is incremented and sent upwards to the controller 21a. The error handler 121a checks for increments, sends an interrupt signal to all controllers 41a of the movable cart 60, and sends the increment counter back down to the error handler 141a of the problematic robotic arm 40. The child error handlers 141b-d may not receive a direct confirmation that the system-level message was received. Rather, since the child process is receiving commanded states from the controller 41a, the controller 41a keeps track of the counters and releases system-level error states appropriately. In embodiments, the error handlers 141b-d may also receive confirmation that the system-level messages were received.

The error handler 141a checks for subsystem interrupt counter increments, whereas the error handler 121a checks for system interrupt counter increments. The child error handler 141b-d, which detected an error continually marks error states as true for several ticks until the subsystem and system counters match. This system of counters forms a cycle which clears the pipeline of nominal commands. It also prevents a user from being able to command unavailable functionality in the window of time between an error being detected and the interrupt/error state signals reaching their destinations.

Error handlers 121a-b and 141a-d in all subsystems send messages to ANS 150 and audio player subsystem 200 about what conditions rise and fall. Each condition or other notification event has a unique alarm ID and ANS 150 and audio player subsystem 200 knows which movable cart 60 and/or the robotic arm 40 originates the alarm. The ANS 150 includes a database storing, for each alarm ID, a notification type, priority, and message and duration of the notification along with an identification of any users being particularly relevant to the specific notification event if applicable. Likewise, the audio player subsystem 200 includes a database storing, for each alarm ID, a corresponding action to take against the audio being played (e.g., adjust playback speed, change song being played, adjust playback volume, adjust playback tone, stop or pause playback, etc.) and a list of relevant users associated with the alarm ID of the notification event. The subsystem error handlers 141a-d include a database storing for each alarm ID corresponding controller reaction types and durations for the reaction.

With respect to dismissible errors, since the error handlers 121a-b and 141a-d are not aware of notification dismissals, the ANS 150 outputs a position control blocking signal which is sent to the controller 21a. The position control blocking signal is false by default. When a dismissible error occurs, ANS 150 sets the blocking signal to true and the error handler 121a uses this signal to make position control unavailable for all robotic arms 40. Even if the dismissible error did not occur in the controller 21a, the blocking signal from the ANS 150 to the error handler 121a causes the controller 21a to command nominal hold signals to all lower-level state machines.

Alarm IDs are set and cleared through ANS 150. Alarms are set upon occurrence of certain conditions (e.g., the existence of an error or a notification event) and alarms may be cleared upon the condition ending or other circumstances. If the alarm is related to recoverable dismissible errors, then those alarms are cleared immediately after being set, because the ANS 150 utilizes the clear signal to enable a “dismiss” button (not shown) on a notification user interface displayed on one or more of the displays 23, 32, 34, 69 and the user's attention is brought to the “dismiss” button (not shown) by virtue of the adjustment to the playing audio (e.g., music) being played by the audio player subsystem 200. Without clearing the alarm through ANS 150, the user will not be able to dismiss the notification, and the error will not be transient.

For persistent dismissible errors, the alarms clear upon condition ending. Thus, the ANS 150 does not enable the “dismiss” button on the notification user interface until the condition ends, at which point the user is allowed to dismiss the notification and the ANS 150 stops blocking teleoperation by the controller 21a. An error signal mapped to a transient dismissible error may still be present, but the controller 21a is able to command position control again as soon as the user acknowledges the notification. To bring the user's attention to the ability to select the “dismiss” button (e.g., upon enablement of the “dismiss” button when the condition ends), the audio player subsystem 200 may adjust the audio being played when the “dismiss” button becomes enabled and selectable.

Each of the controller 21a and the controller 41a have an error aggregator 221a and 241a respectively, which aggregate errors from non-controller software in their respective domains, and forward those errors to the corresponding controller 21a or 41a. Error aggregators 221a and 241a may be embodied as software applications executable by each of the corresponding controllers 21a and 41a, respectively. The error aggregator 221a aggregates errors from all nodes of the control tower 20 and the surgical console 30 and forwards those errors to the controller 21a. The error aggregator 241a aggregates errors from non-controller software on each movable cart 60 and forwards those errors to the main cart controller 41a. The error aggregators 221a and 241a forward the errors to the error handlers 141b-d in the form of an array, which includes a counter of all active errors of a specific system and subsystem including their recoverability type. Upon receiving this array, the error handlers 141b-d identify any new errors that have occurred in the form of an increase in any of the counters, and also for any active errors in the form of non-zero counter values. These two conditions are used by the error handlers 141a-d to create the appropriate controller responses. The error handler response is the same as the response as if the error occurred in the respective controller that received the error report.

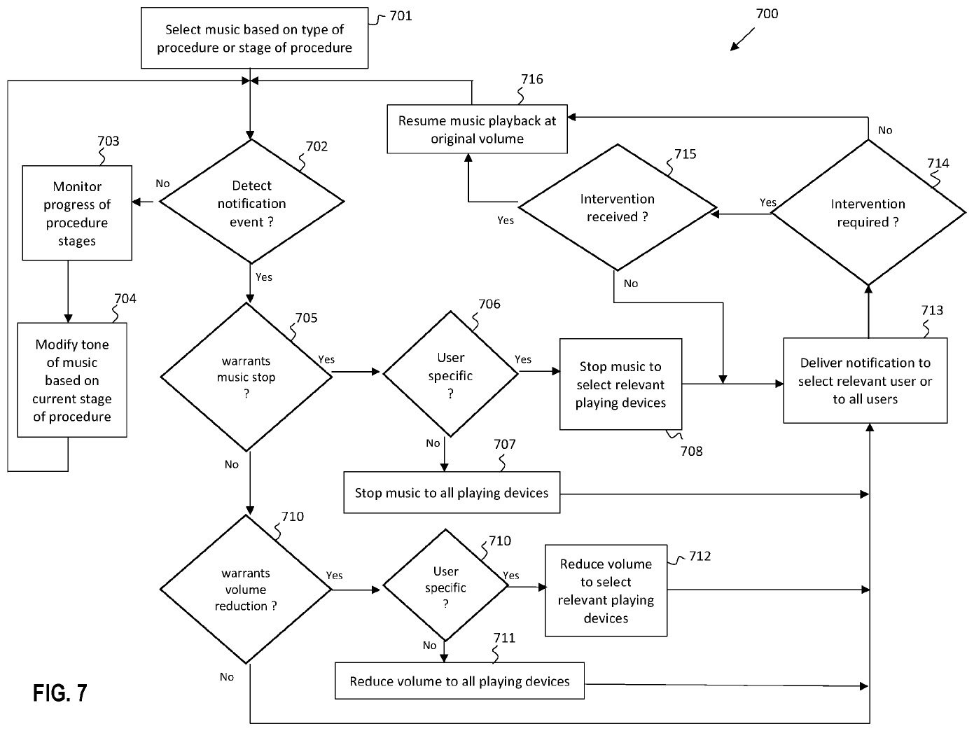

With reference to FIG. 7, a method for controlling audio in a surgical robotic system 10 will now be described as method 700. Method 700 may be embodied as software instructions executed by the computers 21, 31, 41 or any other computing device and may be carried out by one or more components of the surgical robotic system 10. Additionally, although method 700 is described as including particular steps and as being carried out in a particular order, it is contemplated that method 700 may include more or less steps than described and may be carried out in any order.

Method 700 begins in step 701 where particular music is selected for playback. As described above, audio player subsystem 200 (FIG. 5) may store, or may be communicatively coupled to a storage device that stores, a plurality of music audio files. In embodiments, the audio files may be stored remotely on a server, a cloud service, or may be provided by a music streaming service. In step 701, audio player subsystem 200 selects a particular audio file to play based on the type of procedure to be performed or based on a particular stage of the procedure. For example, if the procedure stage is at the start of the procedure (e.g., during preparation and set up), then audio player subsystem 200 may select a song that is suitable for the setup stages of a procedure.

As the procedure progresses and the media is being played back during the procedure, the surgical robotic system 10 constantly monitors its status and the status of the surgical setting and in step 702 determines whether a notification event is detected (e.g., error detection). If in step 702, a notification event is not detected, then method 700 proceeds to step 703 where the progression of the stages of the procedure are monitored. In step 704, the audio player subsystem 200 modifies the tone of the music and/or another setting of the music playback based on the current stage of the procedure (as monitored in step 703).

If in step 702, a notification event is detected, then method 700 proceeds to step 705 where a determination is made whether the notification event warrants a stop of the music playback. In particular, the audio player subsystem 200 looks up the alarm ID data corresponding to the notification event detected in step 702 and determines whether the notification event warrants a stop of the music play for any of the users based on the data associated with the alarm ID. This may be accomplished by stopping the music playback to audio output devices 250 associated with specific users or by redirecting the directional soundwaves emitted by a directional audio output device 250 toward the specific users only and away from the nonrelevant users. If the notification event does warrant a stop of music play for any of the users, then method 700 proceeds to step 706, where a determination is made whether the notification event is relevant to any specific users within the surgical setting. In particular, the audio player subsystem 200 looks up the alarm ID data corresponding to the notification event detected in step 702 at determines whether the notification event is relevant to all of the users or only a select number of users based on the data associated with the alarm ID.

If in step 706 it is determined that the notification event is relevant to all of the users, then, in step 707, the audio player subsystem 200 stops the music playing to all of the users. Stopping the music may include pausing or muting the playback of the music. Alternatively, if in step 706 it is determined that the notification event is relevant to only a select number of the users, then, in step 708, the audio player subsystem 200 stops the music playing to only the relevant users and continues playing music to the other users within the surgical setting, for example by stopping the music playback to audio output devices 250 associated with specific users or by redirecting the directional soundwaves emitted by a directional audio output device 250 toward the specific users only and away from the nonrelevant users. The audio player subsystem 200 may reduce stop the music play for a preset period of time, and then continue the music play, based on the specific notification event and the alarm ID data and whether the notification event requires user acknowledgement or intervention.

If in step 705, it is determined that the notification event does not warrant a stop of the music playback, then method 700 proceeds to step 709, where a determination is made whether the notification event warrants a volume reduction. In particular, the audio player subsystem 200 looks up the alarm ID data corresponding to the notification event detected in step 702 at determines whether the notification event warrants a volume reduction of the music play for any of the users based on the data associated with the alarm ID. If the notification event does warrant a volume reduction of music play for any of the users, then method 700 proceeds to step 710, where a determination is made whether the notification event is relevant to any specific users within the surgical setting. In particular, the audio player subsystem 200 looks up the alarm ID data corresponding to the notification event detected in step 702 at determines whether the notification event is relevant to all of the users or only a select number of users based on the data associated with the alarm ID. If in step 710 it is determined that the notification event is relevant to all of the users, then, in step 711, the audio player subsystem 200 reduces the volume of the music playing to all of the users. Alternatively, if in step 710 it is determined that the notification event is relevant to only a select number of the users, then, in step 712, the audio player subsystem 200 reduces the volume of the music playing to only the relevant users and continues playing music without a reduction in volume to the other users within the surgical setting, for example by reducing the volume of the music playback to audio output devices 250 associated with specific users or by redirecting the directional soundwaves emitted by a directional audio output device 250 toward the specific users only and away from the nonrelevant users. The audio player subsystem 200 may reduce the volume for a preset period of time, and then raise to the original volume level, based on the specific notification event and the alarm ID data and whether the notification event requires user acknowledgement or intervention.

After any of steps 707, 708, 711, or 712, method 700 proceeds to step 713 where a notification is sent to either all of the users or only relevant users depending on the type of notification event and the alarm ID data. The notification delivered may be tactile, audible, vibratory, visual, and/or any other type of notification. In step 714, a determination is made whether user intervention is required to address the notification event based on the alarm ID data of the notification event. User intervention may include acknowledging the notification event (for example, by selecting a button displayed on a user interface such as a “dismiss” button) and/or requiring a correction of the issue that has caused the notification event. If in step 714 it is determined that the notification event requires user intervention, then method 700 proceeds to step 715 where it is determined whether the required user intervention is received. If after a preset period of time (which may be different based on the type of notification event and the alarm ID data associated therewith), the required user intervention is not received, then method 700 reverts back to step 713 where the delivery of the notification is continued. However, if in step 714 it is determined that user intervention is not required, or in step 715 it is determined that the required user intervention is received, then method 700 proceeds to step 716 where the music playback is resumed to original volume levels.

It will be understood that various modifications may be made to the embodiments disclosed herein. In embodiments, the sensors may be disposed on any suitable portion of the robotic arm. Therefore, the above description should not be construed as limiting, but merely as exemplifications of various embodiments. Those skilled in the art will envision other modifications within the scope and spirit of the claims appended thereto.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A surgical robotic system comprising:
a robotic arm including a surgical instrument coupled thereto;
a surgical console including:
a handle communicatively coupled to the robotic arm; and
a display device configured to display a user interface;
a control tower communicatively coupled to the robotic arm and the surgical console;
an error handler communicatively coupled to at least one of the robotic arm, the surgical console, or the control tower and configured to detect a notification event; and
an audio player subsystem communicatively coupled to the error handler, wherein the audio player subsystem is configured to:
play an audio file through an audio output device operably coupled to the audio player subsystem;
receive a detection of a notification event from the error handler; and
control playback of the audio file through the audio output device based on the detection of the notification event.

2. The surgical robotic system according to claim 1, wherein the audio player subsystem is configured to control playback of the audio file through the audio output device based on the detection of the notification event by at least one of stopping, pausing, or muting the playback of the audio file.

3. The surgical robotic system according to claim 2, wherein the audio player subsystem is configured to:
receive a user acknowledgment of the detection of the notification event; and
at least one of restart, un-pause, or un-mute the playback of the audio file upon receipt of the user acknowledgment.

4. The surgical robotic system according to claim 1, wherein the audio player subsystem is configured to control playback of the audio file through the audio output device based on the detection of the notification event by decreasing a volume level of the playback of the audio file.

5. The surgical robotic system according to claim 4, wherein the audio player subsystem is configured to:
receive a user acknowledgment of the detection of the notification event; and
increase the volume level of the playback of the audio file upon receipt of the user acknowledgment.

6. The surgical robotic system according to claim 1, wherein the audio output device is a directional audio output device configured to generate directional sound waves configured to be targeted in a direction of a specific user associated with the notification event.

7. The surgical robotic system according to claim 1, further comprising a second audio output device operably coupled to the audio player subsystem, wherein the audio player subsystem is configured to control playback of the audio file to the audio output device and the second audio output device based on the detection of the notification event.

8. The surgical robotic system according to claim 7, wherein the audio player subsystem is configured to control playback of the audio file through the audio output device and the second audio output device based on the detection of the notification event by adjusting the playback of the audio file through the audio output device in a first manner and adjusting the playback of the audio file through the second audio output device in a second manner, the first manner being different from the second manner.

9. The surgical robotic system according to claim 1, further including an alarm and notification subsystem coupled to the error handler, the alarm and notification subsystem configured to display a notification on the display device based on the detection of the notification event from the error handler.

10. A surgical robotic system comprising:
a video analysis subsystem configured to:
detect a notification event within a surgical setting; and
detect a type of surgical procedure or a stage of a surgical procedure; and
an audio player subsystem communicatively coupled to the video analysis subsystem, wherein the audio player subsystem is configured to:
receive the detected type of surgical procedure or stage of the surgical procedure from the video analysis subsystem;
select an audio file from a plurality of audio files to play based on at least one of the detected type of surgical procedure or stage of surgical procedure as detected by the video analysis subsystem;
play the selected audio file through an audio output device operably coupled to the audio player subsystem;
receive a detection of a notification event from the video analysis subsystem; and
control playback of the audio file through the audio output device based on the detection of the notification event by at least one of:
stopping the playback of the audio file,
pausing the playback of the audio file,
muting a volume level of the playback of the audio file, or
decreasing the volume level of the playback of the audio file.

11. The surgical robotic system according to claim 10, wherein the audio player subsystem is configured to:
receive a user acknowledgment of the detection of the notification event; and
control playback of the audio file through the audio output device based on the received user acknowledgment by at least one of:
restarting the playback of the audio file,
un-pausing the playback of the audio file,
un-muting the playback of the audio file, or
increasing the volume level of the playback of the audio file.

12. The surgical robotic system according to claim 10, wherein the audio output device is a directional audio output device configured to generate directional sound waves configured to be targeted in a direction of a specific user associated with the notification event.

13. The surgical robotic system according to claim 10, further comprising a second audio output device operably coupled to the audio player subsystem, wherein the audio player subsystem is configured to control playback of the audio file to the audio output device and the second audio output device based on the detection of the notification event.

14. The surgical robotic system according to claim 13, wherein the audio player subsystem is configured to control playback of the audio file through the audio output device and the second audio output device based on the detection of the notification event by adjusting the playback of the audio file through the audio output device in a first manner and adjusting the playback of the audio file through the second audio output device in a second manner, the first manner being different from the second manner.

15. The surgical robotic system according to claim 10, further including an alarm and notification subsystem configured to cause a display device to display a notification based on the detection of the notification event from the video analysis subsystem.

16. An audio player subsystem of a surgical robotic system, the audio player subsystem configured to:
play an audio file through an audio output device operably coupled to the audio player subsystem;
receive priority alarm information; and
control playback of the audio file through the audio output device based on the received priority alarm information by at least one of:
stopping the playback of the audio file,
pausing the playback of the audio file,
muting a volume level of the playback of the audio file, or
decreasing the volume level of the playback of the audio file.

17. The audio player subsystem according to claim 16, wherein the audio player subsystem is configured to at least one of:
restart, un-pause, or un-mute the playback of the audio file upon at least one of receipt of a user acknowledgment of the received priority alarm information or expiration of a preset period of time; or
increase the volume level of the playback of the audio file upon at least one of receipt of a user acknowledgment of the received priority alarm information or expiration of a preset period of time.

18. The audio player subsystem according to claim 16, wherein the audio output device is a directional audio output device configured to generate directional sound waves configured to be targeted in a direction of a specific user associated with the received priority alarm information.

19. The audio player subsystem according to claim 16, further comprising a second audio output device operably coupled to the audio player subsystem, wherein the audio player subsystem is configured to control playback of the audio file to the audio output device and the second audio output device based on the received priority alarm information by adjusting the playback of the audio file through the audio output device in a first manner and adjusting the playback of the audio file through the second audio output device in a second manner, the first manner being different from the second manner.

20. The audio player subsystem according to claim 16, further comprising a video analysis subsystem configured to detect a stage of the surgical procedure, wherein the audio player subsystem is configured to control playback of audio based on the detected stage of the surgical procedure.
